(19) United States
(12) Patent Application Publication
(10) Pub. No.: US 2022/0211860 A1
(43) Pub. Date: Jul. 7, 2022

(54) PEPTIDE DERIVATIVES AND CONJUGATES THEREOF FOR TREATING CANCER

(71) Applicant: The University of Sydney, Sydney (AU)

(72) Inventors: Pegah Varamini, Sydney (AU); Sepideh Khazeni, Sydney (AU)

(21) Appl. No.: 17/608,463

(22) PCT Filed: Apr. 30, 2020
(86) PCT No.: PCT/AU2020/050429

§ 371 Date: Nov. 2, 2021

(30) Foreign Application Priority Data
May 2, 2019 (AU) 2019901500

(51) Int. Cl. A61K 47/64; A61K 47/65; A61K 47/55; A61P 35/00

(52) U.S. Cl. CPC A61K 47/6415; A61K 47/64; A61P 35/00; A61K 47/552; A61K 47/65

(57) ABSTRACT

Provided herein are luteinizing hormone-releasing hormone (LHRH) peptide derivatives that target the LHRH receptor. LHRH peptide derivatives, LHRH peptide-drug conjugates (LHRH-PDCs) and methods of using the derivatives and/or conjugates thereof to treat a LHRH receptor expressing cancer are described.

### FIELD OF THE INVENTION

The present invention relates to LHRH peptide derivatives that target the luteinizing hormone-releasing hormone (LHRH) receptor. In particular, LHRH peptide derivatives, LHRH peptide-drug conjugates (LHRH-PDCs), and methods of using the derivatives and/or conjugates thereof to treat a LHRH receptor expressing cancer are described.

### BACKGROUND OF THE INVENTION

Any discussion of the prior art throughout the specification should in no way be considered as an admission that such prior art is widely known or forms part of common general knowledge in the field.

Triple-negative breast cancer (TNBC) constitutes 15% to 20% of all breast cancers and is immunohistochemically defined by the lack of estrogen receptor (ER), progesterone receptor (PR) and human epidermal growth factor receptor 2 (HER2) expression (A. Diana et al., Curr. Oncol. Rep., 2008, 20; A C. Garrido-Castro et al., Cancer Discov., 2019, 9). While TNBC is often more chemo-sensitive than other types of breast cancer, it is a more aggressive form with a high prevalence in younger women and a risk of relapse within the first 3 to 5 years after completion of adjuvant chemotherapy. There has been limited therapeutic progress for treating TNBC in the past several decades and chemotherapy is still the standard of care. There is thus an urgent unmet need for the development of targeted therapeutics with increased selectivity and efficacy, and decreased toxicity for this patient population.

Targeted therapy can be achieved through targeted delivery systems, mainly antibody-drug conjugates (ADCs) and peptide drug conjugates (PDCs) (J M. Reichert, MAbs, 2011, 3). ADCs in general are associated with a number of drawbacks including high cost of production, structural heterogenicity, low coupling ratio of antibody to the cytotoxic drug, and limited tumour penetration (MA. Firer & G. Gellarman, J Hematol. Oncol., 2012, 5). The LHRH receptors are expressed on various tumours including breast, ovarian, endometrial, prostatic, bladder and pancreatic. For example, about 74% of TNBCs express receptors for LHRH, making ligands for this receptor potential carriers to deliver cytotoxic agents directly to these cancerous cells (C. Fost, Oncol. Rep., 2011, 25). While a LHRH-conjugated-doxorubicin has reached clinical trials for prostate cancer, this PDC failed in Phase III clinical trials. Currently, there is still no targeted therapy available for TNBC patients. Thus, there is a need for targeted delivery systems for treating cancer patients, particularly TNBC patients.

It is an object of the present invention to overcome or ameliorate at least one of the disadvantages of the prior art, or to provide a useful alternative.

### SUMMARY OF THE INVENTION

The invention relates to LHRH peptide derivatives and/or LHRH peptide-drug conjugates (LHRH-PDCs). LHRH peptide derivatives described herein exhibit high affinity for their receptor. The LHRH peptide derivatives described herein have improved half-lives compared to the native LHRH peptide and known LHRH agonists, such as triptorelin ([w⁶] LHRH) and [k⁶] LHRH.

In some embodiments, the LHRH peptide derivatives have high enzymatic stability. In certain embodiments, the LHRH peptide derivatives have high shelf stability.

LHRH peptide derivatives as described herein exhibit direct anti-proliferative activity. In certain embodiments, the LHRH peptide derivative shows higher anti-proliferative activities compared to both the native LHRH and the agonist [w⁶] LHRH in breast cancer cell lines including the TNBC cell model. As described herein, the LHRH peptide derivatives of the invention are LHRH receptor agonists.

Provided are LHRH peptide derivatives conjugated to a cytotoxic agent via a linker or fused to a cytotoxic agent.

Provided are methods for treating LHRH receptor expressing cancers comprising administering a LHRH peptide derivative and/or a LHRH peptide-drug conjugate (LHRH-PDCs). In particular, provided are methods of treating TNBC comprising administering a LHRH peptide derivative and/or LHRH-PDC.

Provided is a therapy for cancer comprising administering a LHRH peptide derivative and/or LHRH-PDC. Exemplary cancers that may be treated include, but is not limited to, targeting a LHRH receptor expressing cancer, for example, breast cancer, prostate cancer, colon cancer, ovarian cancer, endometrial cancer, and the like. In particular, provided are methods to target a LHRH receptor expressing cancer cell in TNBC.

In a first aspect, the invention provides a LHRH peptide-drug conjugate (LHRH-PDC) comprising the sequence:

X₁-His-Trp-Ser-X₂-X₃(L-D)-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys;
R is CH₂CH₃ or CH₃;
wherein
L is a linker; and
D is a cytotoxic agent,
   or a pharmaceutically acceptable salt thereof. In certain embodiments, X₁ is Gln, X₃ is D-Lys and R is CH₂CH₃. In some embodiments, X₁ is pGlu, X₃ is D-Lys and R is CH₂CH₃. In some embodiments, X₁ is pGlu, X₃ is D-Lys(Ahx) and R is CH₂CH₃. In certain embodiments, X₁ is Gln, X₂ is Tyr, X₃ is D-Lys, X₄ is Leu, X₅ is Arg and R is CH₂CH₃. In certain embodiments, X₁ is pGlu, X₂ is Tyr, X₃ is D-Lys, X₄ is Leu, X₅ is Arg and R is CH₂CH₃. In certain embodiments, X₁ is pGlu, X₂ is Tyr, X₃ is D-Lys(Ahx), X₄ is Leu, X₅ is Arg and R is CH₂CH₃. In certain embodiments, the linker is a cleavable linker, for example, dipeptide-based linkers with/without PAB (p-aminobenzyl alcohol) or non-peptide cleavable linkers, such as glucuronide linkers which incorporate a hydrophilic sugar group cleaved by b-glucuronidase. In some embodiments, the linker is an uncleavable linker. In some embodiments, the cleavable linker is a self-immolative linker. In related embodiments, the self-immolative linker is maleimidocaproyl valine-citrulline-p-aminobenzyl carbamoyl (mc-vc-PABC). In certain embodiments, the cytotoxic agent is an anti-mitotic agent, an alkylating agent, an anti-metabolite, a topoisomerase inhibitor or a protein kinase inhibitor. In some embodiments, the cytotoxic agent monomethyl auristatin E (MMAE).

In certain embodiments, a LHRH peptide derivative may be fused with a cytotoxic agent. In a related embodiment, a LHRH peptide derivatives is fused with a cytotoxic agent via an uncleavable linker.

In a second aspect, the invention provides an anti-proliferative LHRH peptide derivative comprising the sequence:

X₁-His-Trp-Ser-X₂-X₃-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys; and
R is CH₂CH₃ or CH₃,
   or a pharmaceutically acceptable salt thereof. In certain embodiments, X₁ is Gln, X₃ is D-Lys and R is CH₂CH₃. In some embodiments, X₁ is pGlu, X₃ is D-Lys and R is CH₂CH₃. In some embodiments, X₁ is pGlu, X₃ is D-Lys(Ahx) and R is CH₂CH₃. In certain embodiments, X₁ is Gln, X₂ is Tyr, X₃ is D-Lys, X₄ is Leu, X₅ is Arg and R is CH₂CH₃. In certain embodiments, X₁ is pGlu, X₂ is Tyr, X₃ is D-Lys, X₄ is Leu, X₅ is Arg and R is CH₂CH₃. In certain embodiments, X₁ is pGlu, X₂ is Tyr, X₃ is D-Lys(Ahx), X₄ is Leu, X₅ is Arg and R is CH₂CH₃.

In a third aspect, the invention provides a LHRH peptide derivative comprising the sequence: Gln-His-Trp-Ser-Tyr-D-Lys-Leu-Arg-Pro-NHCH₂CH₃ or a pharmaceutically acceptable salt thereof.

In a fourth aspect, the invention provides a LHRH peptide derivative comprising the sequence: pGlu-His-Trp-Ser-Tyr-D-Lys-Leu-Arg-Pro-NHCH₂CH₃ or a pharmaceutically acceptable salt thereof.

In a fifth aspect, the invention provides a LHRH peptide derivative comprising the sequence: pGlu-His-Trp-Ser-Tyr-D-Lys(Ahx)-Leu-Arg-Pro-NHCH₂CH₃ or a pharmaceutically acceptable salt thereof.

In some embodiments, a LHRH peptide derivative has a half-life of at least about 200 min. In certain embodiments, the LHRH peptide derivative has a half-life of about 200 min to about 250 min. In some embodiments, a LHRH peptide derivative has a half-life of about 250 min to about 300 min. In some embodiments, a LHRH peptide derivative has a half-life of about 300 min to about 350 min. In certain embodiments, a LHRH peptide derivative has a half-life of about 365 min.

In some embodiments, a LHRH-PDC comprises a LHRH peptide derivative conjugated to a cytotoxic agent via a self immolative linker. In certain embodiments, a LHRH-PDC comprises a LHRH peptide derivative conjugated to MMAE via a self immolative linker. In certain embodiments, a LHRH-PDC comprises a LHRH peptide derivative conjugated to MMAE via mc-vc-PABC.

In a sixth aspect, the invention provides a LHRH-PDC comprising a LHRH peptide derivative comprising the sequence: Gln-His-Trp-Ser-Tyr-D-Lys-Leu-Arg-Pro-NHCH₂CH₃ or a pharmaceutically acceptable salt thereof, wherein the LHRH peptide derivative is conjugated to MMAE via mc-vc-PABC.

In a seventh aspect, the invention provides a LHRH-PDC comprising a LHRH peptide derivative comprising the sequence: pGlu-His-Trp-Ser-Tyr-D-Lys-Leu-Arg-Pro-NHCH₂CH₃ or a pharmaceutically acceptable salt thereof, wherein the LHRH peptide derivative is conjugated to MMAE via mc-vc-PABC.

In an eighth aspect, the invention provides a LHRH-PDC comprising a LHRH peptide derivative comprising the sequence: pGlu-His-Trp-Ser-Tyr-D-Lys(Ahx)-Leu-Arg-Pro-NHCH₂CH₃ or a pharmaceutically acceptable salt thereof, wherein the LHRH peptide derivative is conjugated to MMAE via mc-vc-PABC.

In a ninth aspect, the invention provides a pharmaceutical composition comprising a LHRH-PDC, or a pharmaceutically acceptable salt thereof, and optionally at least one pharmaceutically acceptable excipient.

In a tenth aspect, the invention provides a pharmaceutical composition comprising a LHRH peptide derivative of the invention, or a pharmaceutically acceptable salt thereof, and optionally at least one pharmaceutically acceptable excipient.

In an eleventh aspect, the invention provides a method of treating cancer in a subject in need thereof, comprising administering to the subject a therapeutically effective amount of a LHRH-PDC or a pharmaceutically acceptable salt thereof, a LHRH peptide derivative or a pharmaceutically acceptable salt thereof, or a pharmaceutical composition of the invention, wherein the subject has a LHRH receptor expressing cancer. In some embodiments, the LHRH receptor expressing cancer is breast cancer, prostate cancer, colon cancer, ovarian cancer, pancreatic cancer or endometrial cancer. In some embodiments, the LHRH receptor expressing cancer is TNBC. In certain embodiments, the method of treating cancer comprises administering a LHRH-PDC wherein the cancer is TNBC.

In a twelfth aspect, the invention provides a method of treating cancer in a subject in need thereof, comprising administering to the subject a therapeutically effective amount of a LHRH-PDC or a pharmaceutically acceptable salt thereof, and optionally at least one pharmaceutically acceptable excipient with one or more additional cytotoxic agent(s), wherein the subject has a LHRH receptor expressing cancer. In some embodiments, the LHRH receptor expressing cancer is breast cancer, prostate cancer, colon cancer, ovarian cancer, pancreatic cancer or endometrial cancer. In some embodiments, the LHRH receptor expressing cancer is TNBC.

In a thirteenth aspect, the invention provides a method of arresting or retarding cell growth and/or proliferation of a LHRH receptor expressing tumour in a subject in need thereof, comprising administering to the subject a therapeutically effective amount of a LHRH-PDC or a pharmaceutically acceptable salt thereof, a LHRH peptide derivative or a pharmaceutically acceptable salt thereof, or a pharmaceutical composition of the invention.

In a fourteenth aspect, the invention provides a method of arresting or retarding cell growth and/or proliferation of a LHRH receptor expressing tumour in a subject in need thereof, comprising administering to the subject a therapeutically effective amount of a LHRH-PDC or a pharmaceutically acceptable salt thereof, and optionally at least one pharmaceutically acceptable excipient and one or more additional cytotoxic agent(s).

In a fifteenth aspect, the invention provides a method of treating a hormone sensitive and/or refractory breast cancer in a subject in need thereof, comprising administering to the subject a therapeutically effective amount of a peptide conjugate of the invention, a LHRH peptide derivative or a pharmaceutical composition of the invention.

In a sixteenth aspect, the invention provides a method of treating a hormone sensitive and/or refractory breast cancer in a subject in need thereof, comprising administering to the subject a therapeutically effective amount of a LHRH-PDCor a pharmaceutically acceptable salt thereof, and optionally at least one pharmaceutically acceptable excipient and one or more additional cytotoxic agent.

In a seventeenth aspect, the invention provides use of a LHRH-PDC or a pharmaceutically acceptable salt thereof, a LHRH peptide derivative or a pharmaceutically acceptable salt thereof, or a pharmaceutical composition of the invention in the manufacture of a medicament for treating cancer, wherein the cancer is a LHRH receptor expressing cancer.

In an eighteenth aspect, the invention provides use of a LHRH-PDC or a pharmaceutically acceptable salt thereof, a LHRH peptide derivative or a pharmaceutically acceptable salt thereof, or a pharmaceutical composition of the invention in the manufacture of a medicament for arresting or retarding cell growth and/or proliferation of a LHRH receptor expressing tumour.

In a nineteenth aspect, the invention provides use of a LHRH-PDC or a pharmaceutically acceptable salt thereof, a LHRH peptide derivative or a pharmaceutically acceptable salt thereof, or a pharmaceutical composition of the invention in the manufacture of a medicament for treating hormone sensitive and/or refractory breast cancer.

In a twentieth aspect, the invention provides use of a LHRH-PDC or a pharmaceutically acceptable salt thereof, a LHRH peptide derivative or a pharmaceutically acceptable salt thereof, or a pharmaceutical composition of the invention in the manufacture of a medicament for treating TNBC.

Methods of synthesizing and/or producing a LHRH peptide derivative and/or LHRH-PDC herein disclosed are not particularly limited and any suitable method may be used.

### Definitions

Unless the context clearly requires otherwise, throughout the description and the claims, the words “comprise”, “comprising” and the like are to be construed in an inclusive sense as opposed to an exclusive or exhaustive sense; that is to say, in the sense of “including, but not limited to”.

As used herein, the singular forms “a,” “an” and “the” refer to “one or more” when used in this application. Thus, for example, reference to “a sample” includes a plurality of such samples, and so forth.

As used herein the term “about” can mean within 1 or more standard deviation per the practice in the art. Alternatively, “about” should be assumed to be within an acceptable error range for that particular value. For example, in the context of half-life values, the term “about” can mean a range of up to 10%.

The term “luteinizing hormone releasing hormone” or “LHRH” is also known as “gonadotrophin-releasing hormone (GnRH)” or “Luteinizing Hormone-Releasing Factor (LRF)” in the art. LHRH is translated from the mRNA as a pro-hormone which is then converted to the mature decapeptide with the native sequence pGlu-His-Trp-Ser-Tyr-Gly-Leu-Arg-Pro-Gly-NH₂. All amino acids in the native LHRH peptide are in their L-form.

The term “peptide” used herein, includes but is not limited to, two or more amino acids, or residues covalently linked by an amide bond or equivalent. In certain embodiments, amino acids may be linked by non-natural and non-amide chemical bonds including but not limited to D-Lys, Pro-Et or Ahx.

The term “amino acid” includes well known amino acids, for example, alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). The above amino acid three letter abbreviations or one letter abbreviations are known and standard in the art. The amino acid described herein, with the exception of Gly, may be in the “L” or “D” stereoisomeric form. The stereoisomeric form may be designated by including “D” or “L” with a standard three letter abbreviation or one letter abbreviation, for example D-Lys and L-Lys. An amino acid in an upper case one-letter code denotes that the amino acid is in its L-form while an amino acid in a lower case one-letter code denotes that the amino acid is in its D-form. In the absence of a “D” or “L” designation, an amino acid in the three letter abbreviation is in the “L” form. Non-traditional amino acids are also within the scope of the invention and include norleucine, ornithine, norvaline, homoserine, and other amino acid analogues. Preferably, the non-traditional amino acids are used in place of D-Lys. The term amino acid residue refers to an amino acid included in a peptide. It will be appreciated that an amino acid residue may be on the N- or C-terminus of a peptide.

The term “pGlu” or “pyroGlu” is known in the art and refers to L-pyroglutamic acid.

The term “NHEt” is known in the art and refers to N-ethylamide. It is also known as ethylmaleimide or NEM.

The term “Ahx” is known in the art and refers to 6-aminohexanoic acid.

The term “LHRH peptide derivative” or “peptide derivative” are used interchangeably herein and refer to a native LHRH peptide comprising at least two modifications, wherein the at least two modifications comprise substitution of the sixth amino acid residue Gly to D-Lys or D-Lys(Ahx) and substitution of the tenth amino acid residue Gly to NHR, wherein R is herein defined. A LHRH peptide derivative may comprise two or more modifications, wherein at least two modifications comprise substitution of the sixth amino acid residue Gly to D-Lys or D-Lys(Ahx) and substitution of the tenth amino acid residue Gly to NHR, wherein R is herein defined, and wherein a further modification comprises substitution of the first amino acid residue pGlu to Gln. In some embodiments, the LHRH peptide derivatives may include additional modifications at amino acid residue positions five, seven, and/or eight.

As used herein, the terms “conjugate”, “peptide conjugate” or “LHRH peptide-drug-conjugate (LHRH-PDC)” are used interchangeably and denote a molecule comprising a LHRH peptide derivative conjugated to a cytotoxic agent via a linker.

The term “linker” denotes a moiety whose purpose is to connect or link, covalently, a cell targeting enhancing moiety, for example a LHRH peptide derivative and a cytotoxic agent. The linker as used herein may be a cleavable or uncleavable linker. In the context of a LHRH peptide derivative herein described, a linker is covalently bonded to a LHRH peptide derivative via the sixth amino acid residue (D-Lys or D-Lys(Ahx)) or at the C- or N-terminus.

As used herein, the term “cytotoxic agent” includes but is not limited to an anti-mitotic agent, an alkylating agent, an anti-metabolite, a topoisomerase inhibitor or a protein kinase inhibitor. The cytotoxic agent may be a vinca alkaloid, a cryptophycin, bortezomib, thiobortezomib, a tubulysin, aminopterin, rapamycin, paclitaxel, docetaxel, daunorubicin, everolimus, a-amanatin, vemcarin, didemnin B, geldanomycin, purvalanol A, ispinesib, budesonide, dasatinib, an epothilone, a maytansine, doxorubicin, camptothecin, methotrexate (MTX) or monomethyl auristatin E (MMAE). In certain embodiments, the cytotoxic agent is MMAE.

Methods of preparing a LHRH peptide derivatives and/or LHRH-PDC are not particularly limiting. Exemplary methods include Fmoc solid-phase chemistry and click chemistry. It will be appreciated that a LHRH peptide derivative may be commercially sourced. It will further be appreciated that commercially available kits may be available for conjugating a cytotoxic agent, for example MMAE, to a LHRH peptide derivative described herein.

Also contemplated are pharmaceutically acceptable salts of a LHRH peptide derivative and/or LHRH-PDC. The term “pharmaceutically acceptable salt” includes both acid and base addition salts and refers to salts which retain the biological effectiveness and properties of the free bases or acids, and which are not biologically or otherwise undesirable. The pharmaceutically acceptable salts are formed with inorganic or organic acids or bases and can be prepared in situ during the final isolation and purification of the compounds, or by separately reacting a purified compound in its free base or acid form with a suitable organic or inorganic acid or base, and isolating the salt thus formed.

As used herein “pharmaceutical composition” or “composition” refers to a mixture of at least one LHRH peptide derivative or LHRH-PDC, or pharmaceutically acceptable salts, solvates, hydrates thereof, with other chemical components, such as pharmaceutically acceptable excipients. Pharmaceutical compositions suitable for the delivery of peptide derivatives or conjugates as described herein and methods for their preparation will be apparent to those skilled in the art.

Also contemplated are pharmaceutical compositions comprising at least one LHRH peptide derivative and/or LHRH-PDC, and optionally at least one pharmaceutical excipient. The term “pharmaceutically acceptable excipient” refers to any pharmaceutically acceptable inactive component of the composition. As is known in the art, excipients include diluents, buffers, binders, lubricants, disintegrants, colorants, antioxidants/preservatives, pH-adjusters, etc. The excipients are selected based on the desired physical aspects of the final form: e.g. a parenteral formulation for injection, obtaining a tablet with desired hardness and friability being rapidly dispersible and easily swallowed, and the like. Suitable forms of a pharmaceutical composition may include, but is not limited to, a tablet, capsule, elixir, liquid formulation, delayed or sustained release, and the like. The physical form and/or content of a pharmaceutical composition contemplated are conventional preparations that may be formulated by those skilled in the pharmaceutical formulation field.

A cancer described herein as expressing a LHRH receptor, includes a cancer cell population that is tumorigenic, including benign tumours and malignant tumours, or non-tumorigenic. Methods of determining LHRH receptor expression in cancer are not particularly limiting. Exemplary methods include western blotting, immunocytochemistry, flow cytometry, and PCR (polymerase chain reaction). Exemplary cancers include but are not limited to a carcinoma, a sarcoma, a lymphoma, a melanoma, a mesothelioma, a nasopharyngeal carcinoma, a leukaemia, an adenocarcinoma, and a myeloma. In certain embodiments, the cancers may be lung cancer, bone cancer, pancreatic cancer, skin cancer, cancer of the head, cancer of the neck, cutaneous melanoma, intraocular melanoma uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer or adenocarcinoma of the gastroesophageal junction.

It is also contemplated that a LHRH peptide derivative and/or LHRH PDC may be delivered to a cancer cell in-vitro or in-vivo. In some embodiments, a LHRH-PDC is administered to a cancer cell in-vitro or in-vivo. In certain embodiments, a LHRH peptide derivative is administered to a cancer cell in-vitro or in-vivo and exhibits anti-proliferative activity. A LHRH peptide derivative and/or LHRH-PDC may be administered to a cell with a pharmaceutically acceptable carrier within a composition as herein described.

As used herein, the term “tumour” refers to neoplastic cell growth and proliferation, whether malignant or benign, and pre-cancerous and cancerous cells and tissues. For example, a particular cancer may be characterized by a solid mass tumour. The term “tumour” is inclusive of solid tumours and non-solid tumours. A LHRH peptide derivative, LHRH-PDC, or composition herein described may be administered to the subject locally at the site of a tumour (e.g., by direct injection) or remotely (e.g., systemic administration). In some embodiments, A LHRH peptide derivative, LHRH-PDC, or composition herein described may be administered to the subject systemically, e.g., intravascular, such as intravenous administration.

The term “anti-proliferative activity” refers the ability of a compound to stop the growth of cells.

A “subject” to be treated by a method described herein includes mammal, including a human (“patient”) or non-human subject (for example, cat, dog, and the like). A LHRH peptide derivative, LHRH-PDC, or composition herein described may be administered to a human or non-human subject. A LHRH peptide derivative, LHRH-PDC, or composition herein described may be administered to a human cancer cell or a non-human cancer cell in vitro or in vivo. In some embodiments, the cell is a mammalian cell.

As used herein, a “therapeutically effective amount” of a LHRH peptide derivative, LHRH-PDC, or composition herein describes includes an amount, when administered (whether as a single dose or as a time course of multiple treatments), promotes disease regression evidenced by a decrease in severity of disease symptoms, an increase in frequency and duration of disease symptom-free periods, or a prevention of impairment or disability due to the disease affliction. A therapeutically effective amount of a LHRH peptide derivative, LHRH-PDC or composition herein described includes a “prophylactically effective amount” which is any amount of a LHRH peptide derivative, LHRH-PDC or composition that, when administered to a subject at risk of developing a disease or of suffering a recurrence of disease, inhibits the development or recurrence of the disease. The ability of a therapeutic agent to promote disease regression or inhibit the development or recurrence of the disease may be evaluated using a variety of methods known to the skilled practitioner, such animal model systems predictive of efficacy in humans, by assaying the activity of the agent in in vitro assays, or the like. By way of example for the treatment of cancer, a therapeutically effective amount of a LHRH peptide derivative, LHRH-PDC or composition as described herein may inhibit cancer cell growth by at least about 20%, by at least about 40%, by at least about 60%, or by at least about 80% relative to untreated cancer cells. In some embodiments, for the treatment of cancer, a therapeutically effective amount of a LHRH peptide derivative as described herein may inhibit cancer cell growth by about 60%. In some embodiments, for the treatment of cancer, a therapeutically effective amount of a LHRH-PDC as described herein may inhibit cancer cell growth by about 80%. A therapeutically effective amount of a LHRH peptide derivative, LHRH-PDC, or composition herein described may completely inhibit cell growth or tumor growth. A therapeutically effective amount of a LHRH peptide derivative, LHRH-PDC, or composition herein described may inhibit or reduce to a statistically significant degree cell growth or tumour growth as compared to control. “Statistical significance” means significance at the p<0.05 level, or such other measure of statistical significance as would be used by those of skill in the art of biomedical statistics in the context of a particular type of treatment or prophylaxis.

Depending upon the cancer type as described herein, the route of administration and/or whether a LHRH peptide derivative, LHRH-PDC, or composition as herein described is administered locally or systemically, a wide range of permissible dosages are contemplated. A suitable dose, includes a doses falling in the range from about 0.5 mg/kg to about 5 mg/kg. The dosages may be single or divided and may be administered according to a wide variety of protocols, including q.d., b.i.d., t.i.d., or even every other day, biweekly (b.i.w.), once a week, once a month, once a quarter, and the like. In each of these cases, it is understood that the therapeutically effective amounts described herein correspond to the instance of administration, or alternatively to the total daily, weekly, month, or quarterly dose, as determined by the dosing protocol.

It is also contemplated that a LHRH peptide derivative, LHRH-PDC, or composition as herein described may be administered with one or more cytotoxic agents. Administration as a LHRH peptide derivative, LHRH-PDC, or composition as herein described with one or more additional cytotoxic agents may include simultaneous administration, or sequential administration. Sequential administration includes an administration regime wherein one or more hours, or one or more days, separate the administration of a LHRH peptide derivative, LHRH-PDC, or composition as herein described and the one or more cytotoxic agents.

As described herein in vitro metabolic stability in plasma is defined as the susceptibility of a chemical compound to biotransformation in plasma, and is expressed as in vitro half-life (T_{1/2}).

### BRIEF DESCRIPTION OF THE FIGURES

Embodiments of the invention will now be described, by way of example only, with reference to the accompanying drawings as follows.

FIGS. 1A and 1B: (A) Structure of exemplary LHRH peptide derivatives: Gln-His-Trp-Ser-Tyr-D-Lys-Leu-Arg-Pro-NHCH₂CH₃ (LD4), pGlu-His-Trp-Ser-Tyr-D-Lys-Leu-Arg-Pro-NHCH₂CH₃ (LD5) and pGlu-His-Trp-Ser-Tyr-D-Lys(Ahx)-Leu-Arg-Pro-NHCH₂CH₃ (LD6). With the exception of D-Lys at the sixth position, all amino acids in the LHRH peptide derivatives of FIG. 1A are present in their L-form. (B) The metabolic stability of LHRH peptide derivatives examined in human plasma and quantified by Liquid Chromatography-Mass-Spectrometry (LC-MS) in a time dependent manner. The LHRH peptide derivatives of the invention exhibit substantially improved half-life (T_{1/2} of about 257 min, 365 min and 309 min for LD4, LD5 and LD6, respectively) compared to the native LHRH peptide (T_{1/2} of about 10 min) and LHRH agonists triptorelin ([w⁶] LHRH) and [k⁶] LHRH (T_{1/2} of about 19 min and 39 min, respectively). Statistical analysis was performed using a one-way ANOVA followed by Dunnett's post-hoc test. Error bars are standard deviations (STDV), p<0.05, n=9 (3 independent experiments each performed in triplicate).

FIG. 2: LHRH receptor (LHRH-R) expression level in 3 breast cancer cell lines (MCF-7, MDA-MB-231 and SK-BR-3) versus 3-actin as the control determined using Western blot analysis. The breast cancer cell lines have different characteristics: MDA-MB-231: ER−, HER-2−, PR−; MCF-7: ER+, HER-2+, PR+; and SK-BR-3: ER−, HER-2+, PR−. A major protein band of approximately 64 kDa molecular mass known for the human pituitary LHRH receptor was identified in MCF-7, MDA-MB-231 and SK-BR-3 (lanes 1, 2, and 3 respectively). The level of LHRH-R expression relative to β-actin in MCF-7, MDA-MB-231 and SK-BR-3 showed that these breast cancer cells can be actively targeted through the LHRH receptor. The Western blot analysis was performed using the antibody specifically raised against the LHRH-R.

FIGS. 3A and 3B: (A) The expression of LHRH-Rs was further confirmed by immunohistochemistry in MDA-MB-231 (TNBC cell model), SKOV-3 (low LHRH-R expressing cell model), HMEC and MCF-10A as normal breast cells. FIG. 3A shows specific receptor binding of primary antibody labelled by Rhodamine-conjugated secondary antibody. (B) The LHRH-R signal intensity was quantified by Fiji J software in normal breast cells (HMEC and MCF-10A), MDA-MB-231 and SKOV-3. The signal intensity in these cell lines were assessed by One-way ANOVA followed by Tukey's multiple comparisons test (****p<0.0001). The LHRH-R expression signal intensity is significantly higher in MDA-MB-231 compared to SKOV-3, HMEC and MCF-10A. Error bars are standard deviations (STDV), n=9 (3 independent experiments each performed in triplicate).

FIG. 4A to 4E: The cytotoxic activity of the peptide conjugate LD5-mc-vc-PABC-MMAE (LM) in comparison to MMAE was assessed in MDA-MB-231 (TNBC cell model) versus HMEC and MCF-10A (normal breast cells) and SKOV-3 (low LHRH-R expressing cell model), by MTT assay after incubating cells with compounds in serial dilution for 72 h. (A) The relative cellular viability was assessed in comparison to PBS-treated negative control group and IC₅₀ value (nM) was calculated by non-linear regression (curve fit) for HMEC and MCF-10A (normal breast cells) and MDA-MB-231 (LHRH-R positive, TNBC cell model) and SKOV-3 (LHRH-R low expressing cells). (B) The cell viability in LD5-me-vc-PABC-MMAE (LM) was assessed in HMEC as normal breast cell line in comparison to MMAE by two-way ANOVA followed by Sidak's multiple comparisons test (****p<0.0001). (C) The cell viability in LD5-mc-vc-PABC-MMAE (LM) was assessed in MCF-10A as normal breast cell line in comparison to MMAE by two-way ANOVA followed by Sidak's multiple comparisons test (***p<0.001, **p<0.01, *p<0.05). (D) The cell viability in LD5-me-vc-PABC-MMAE (LM) was assessed in MDA-MB-231 (TNBC cell model) in comparison to MMAE by two-way ANOVA followed by Sidak's multiple comparisons test (****p<0.0001). (E) The cell viability in LD5-me-vc-PABC-MMAE (LM) was assessed in SKOV-3 (LHRH-R low expressing cells) in comparison to MMAE by two-way ANOVA followed by Sidak's multiple comparisons test (****p<0.0001). LD5-mc-vc-PABC-MMAE (LM) conjugate shows decreased cytotoxicity compared to MMAE due to selectivity of LHRH uptake via the receptor rather than simple diffusion of free MMAE in the cell lines. Error bars are standard deviations (STDV), n=9 (3 independent experiments each performed in triplicate).

FIGS. 5A and 5B: Receptor binding competitive assay. MDA-MB-231 was pre-treated with triptorelin (TRN) and LM (LD5-mc-vc-PABC-MMAE) then the MTT assay was performed. (A) The IC₅₀ value (nM) was calculated by non-linear regression (curve fit) for PC-3 and MDA-MB-231 with and without pre-treatment with triptorelin. (B) The cell viability in TRN-LM was assessed in comparison to LM in MDA-MB-231 (TNBC cell line). The cytotoxic effects of LM is reversed after pre-treatment with triptorelin indicating a significant role of LHRH-Rs in the mechanism of action of LM. Two-way ANOVA was performed followed by Sidak's multiple comparisons test (***p<0.00001). Error bars are standard deviations (STDV), n=8 (3 independent repeats).

FIG. 6: Proximity ligation assay was used to show the protein-protein interaction between LHRH-R and LHRH in MDA-MB-231 (TNBC cell model) treated with LD5-me-vc-PABC-MMAE (1 μM) compared to negative control (PBS). Stained nucleus (DAPI blue) and PLA signal (TexasRed) were detected by LEICA SPE2 confocal LSM.

FIGS. 7A and 7B: In vitro uptake study of LD5-me-vc-PABC-MMAE in TNBC cell line and normal breast cells. (A) MDA-MB-231 as a TNBC cell model, MCF-10A and HMEC as normal breast cells were treated with 1 μM of LD5-me-vc-PABC-MMAE (LM) for 18 h. LM was stained using monoclonal primary antibody specific for LHRH and secondary antibody conjugated with Rhodamine and detected by LEICA SPE2 confocal LSM. (B) The LM signal intensity was measured by Fiji J software in normal breast cells (HMEC and MCF-10A) and TNBC cell model (MDA-MB-231). There was significantly higher uptake of LM in LHRH-R positive cells (MDA-MB-231) compared to normal breast cells. The signal intensity in these cell lines were assessed by one-way ANOVA followed by Tukey's multiple comparisons test (****p<0.0001). Error bars are standard deviations (STDV), n=8 (3 independent repeats).

FIGS. 8A and 8B: The effect of MMAE and LD5-me-vc-PABC-MMAE (LM) on α-tubulin polymerization. (A) MDA-MB-231 (TNBC cell model) and normal breast cells (HMEC and MCF-10A) were treated with free MMAE and LM at 1 μM and PBS (control) followed by immunostaining of α-tubulin with primary and secondary antibodies. The α-tubulin signals were detected by LEICA SPE2 confocal LSM. (B) The α-tubulin signal intensity was measured by Fiji J software in normal breast cells (HMEC and MCF-10A) and MDA-MB-231. ****p<0.0001 is the significance in the signal intensity in each cell line treated with PBS and LM in comparison to MMAE-treated cells. ####p<0.0001 is the significance of the α-tubulin signal intensity in MDA-MB-231 cells treated with LM in comparison to MCF-10A and HMEC cells. The α-tubulin signal in normal breast cells treated with PBS and LM were significantly higher than their MMAE treated counterpart indicating the lower impact of the targeted drug on the α-tubulin polymerization as a factor of cell survival. Statistical analysis was made in Prism by two-way ANOVA followed by Tukey's multiple comparisons test. Error bars are standard deviations (STDV), n=8 (3 independent repeats).

FIGS. 9A and 9B: (A) Cells were co-transfected with LHRH-R siRNAs and fluorescent labelled siRNA negative control as transfection efficiency signal (FAM-transfection control). Negative control refers to MDA-MB-231 cells that were not transfected with LHRH-R siRNAs and FAM-transfection control. LHRH-R expression was detected by immunocytochemistry with specific receptor binding of primary antibody labelled by Rhodamine-conjugated secondary antibody. Immunocytochemical images were captured by LEICA SPE2 confocal LSM. (B) The LHRH-R signal intensity in both silenced LHRH-R and negative control was measured by Fiji J software. Silencing of LHRH-Rs results in significantly reduced LHRH-R expression in transfected MDA-MB-231 cells compared to negative control. The data was analysed by unpaired t-test with Welch's correction comparison between the silenced LHRH-R and negative control (**** p<0.0001). Error bars are standard deviations (STDV), n=8 (3 independent repeats).

FIGS. 10A and 10B: Uptake of LD5-me-vc-PABC-MMAE (LM) after silencing LHRH-R in MDA-MB-231 cells. The cells were treated with LHRH-R siRNA and co-transfected with fluorescent labelled siRNA negative control as transfection efficiency signal (FAM-transfection control). LHRH-R negative control refers to MDA-MB-231 cells that were not transfected with LHRH-R siRNA and FAM-transfection control. (A) Silenced LHRH-R cells and negative control cells were treated with LM (1 μM) for 18 h and LM signal was detected by immunocytochemistry with specific LHRH binding of primary antibody labelled by Rhodamine-conjugated secondary antibody. Immunocytochemical images were captured by LEICA SPE2 confocal LSM. (B) The LM signal intensity in both silenced LHRH-R and negative control was measured by Fiji J software. The uptake of LD5-me-vc-PABC-MMAE was significantly reduced after silencing the LHRH-R gene indicating that the construct is actively targeted to and up-taken by cancer cells via overexpressed LHRH-Rs. The data was analysed by unpaired t-test with Welch's correction (**** p<0.0001). Error bars are standard deviations (STDV), n=8 (3 independent repeats).

FIG. 11: Stability of LHRHD-(mc-vc-PABC)-MMAE (i.e. LD5-mc-vc-PABC-MMAE) in human plasma, mouse plasma, and cell culture media (c-media). The stability was measured based on the presence of free MMAE detected in the samples by LC-MS at each time point relative to time 0. Error Bars are standard deviations (STDV), p>0.05 and n=9 (mean is calculated from samples in triplicate in 3 independent experiments).

### DETAILED DESCRIPTION OF PREFERRED EMBODIMENTS

In one embodiment, provided is a LHRH-PDC comprising the sequence:

X₁-His-Trp-Ser-X₂-X₃(L-D)-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys;
R is CH₂CH₃ or CH₃;
wherein
L is a linker; and
D is a cytotoxic agent,
   or a pharmaceutically acceptable salt thereof. In certain embodiments, X₁ is Gln, X₃ is D-Lys and the R is CH₂CH₃. In some embodiments, X₁ is pGlu, X₃ is D-Lys and R is CH₂CH₃. In some embodiments, X₁ is pGlu, X₃ is D-Lys(Ahx) and R is CH₂CH₃. In certain embodiments, X₁ is Gln, X₂ is Tyr, X₃ is D-Lys, X₄ is Leu, X₅ is Arg and R is CH₂CH₃. In certain embodiments, X₁ is pGlu, X₂ is Tyr, X₃ is D-Lys, X₄ is Leu, X₅ is Arg and R is CH₂CH₃. In certain embodiments, X₁ is pGlu, X₂ is Tyr, X₃ is D-Lys(Ahx), X₄ is Leu, X₅ is Arg and R is CH₂CH₃. In certain embodiments, the linker is a cleavable linker. In some embodiments, the linker is an uncleavable linker. In some embodiments, the cleavable linker is a self-immolative linker. In a related embodiment, the self-immolative linker is maleimidocaproyl valine-citrulline-p-aminobenzyl carbamoyl (mc-vc-PABC). In certain embodiments, the cytotoxic agent is an anti-mitotic agent, an alkylating agent, an anti-metabolite, a topoisomerase inhibitor or a protein kinase inhibitor. In some embodiments, the cytotoxic agent is selected from the group consisting of a vinca alkaloid, a cryptophycin, bortezomib, thiobortezomib, a tubulysin, aminopterin, rapamycin, paclitaxel, docetaxel, daunorubicin, everolimus, a-amanatin, vemcarin, didemnin B, geldanomycin, purvalanol A, ispinesib, budesonide, dasatinib, an epothilone, a maytansine, doxorubicin, camptothecin, methotrexate (MTX) or monomethyl auristatin E (MMAE). In certain embodiments, the cytotoxic agent is MMAE.

In some embodiments, the linker comprises at least one amino acid. In certain embodiments, the linker comprises one or more amino acid residues, wherein the amino acid is one or more of Lys, Asn, Thr, Ser, He, Met, Pro, His, Gin, Arg, Gly, Asp, Glu, Ala, Vai, Phe, Leu, Tyr, Cys, and/or Trp. In some embodiments, the linker comprises a carbon chain, amide bond or ether bond. In some embodiments, the linker comprises a hydrazone bond, vinyl ether bond, acetal bond, ketal bond or disulphide bond. In certain embodiments, the linker comprises Gly-Phe-Leu-Gly. In some embodiments, the linker comprises Val-Cit (Cit=citrulline). In some embodiments, the linker comprises Phe-Lys. In some embodiments, the linker is polyethylene glycol (PEG) chains, acetate linkers, ester linkers, lectins, buSS (disulfylbutyrate) or maleimide.

In a further embodiment, provided is a LHRH-PDC comprising the sequence:

X₁-His-Trp-Ser-X₂-X₃(L-D)-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys;
R is CH₂CH₃ or CH₃;
wherein
L is a linker; and
D is a cytotoxic agent,
   or a pharmaceutically acceptable salt thereof. In some embodiments, X₁ is Gln. In some embodiments, X₁ is pGlu. In some embodiments, X₁ is Gln and X₂ is Tyr. In some embodiments, X₁ is Gln and X₂ is Phe. In some embodiments, X₁ is Gln and X₂ is His. In some embodiments, X₃ is D-Lys. In some embodiments, X₃ is D-Lys(Ahx). In some embodiments, X₁ is Gln and X₄ is Leu. In some embodiments, X₁ is Gln and X₄ is Val. In some embodiments, X₁ is Gln and X₄ is Trp. In some embodiments, X₁ is Gln and X₄ is Met. In some embodiments, X₁ is Gln and X₅ is Arg. In some embodiments, X₁ is Gln and X₅ is Gln. In some embodiments, X₁ is Gln and X₅ is Trp. In some embodiments, X₁ is Gln and X₅ is Ser. In some embodiments, X₁ is Gln and X₅ is Leu. In some embodiments, X₁ is Gln and X₅ is Asn. In some embodiments, X₁ is Gln and X₅ is Phe. In some embodiments, X₁ is Gln and X₅ is Tyr. In some embodiments, X₁ is Gln and X₅ is Lys. In certain embodiments, R is CH₂CH₃. In certain embodiments, R is CH₃.

In certain embodiments, a LHRH peptide derivative may be fused with a cytotoxic agent. In a related embodiment, a LHRH peptide derivatives is fused with a cytotoxic agent via an uncleavable linker.

In a further embodiment, provided is an antiproliferative LHRH peptide derivative comprising the sequence:

X₁-His-Trp-Ser-X₂-X₃-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys; and
R is CH₂CH₃ or CH₃,
   or a pharmaceutically acceptable salt thereof. In certain embodiments, X₁ is Gln, X₃ is D-Lys and R is CH₂CH₃. In some embodiments, X₁ is pGlu, X₃ is D-Lys and R is CH₂CH₃. In some embodiments, X₁ is pGlu, X₃ is D-Lys(Ahx) and R is CH₂CH₃. In certain embodiments, X₁ is Gln, X₂ is Tyr, the X₃ is D-Lys, X₄ is Leu, X₅ is Arg and R is CH₂CH₃. In certain embodiments, X₁ is pGlu, X₂ is Tyr, X₃ is D-Lys, X₄ is Leu, X₅ is Arg and R is CH₂CH₃. In certain embodiments, X₁ is pGlu, X₂ is Tyr, X₃ is D-Lys(Ahx), X₄ is Leu, X₅ is Arg and R is CH₂CH₃.

In a further embodiment, provided is a LHRH peptide derivative comprising the sequence:

X₁-His-Trp-Ser-X₂-X₃-X₄-X₅-Pro-NHR
wherein
X₁ is Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys; and
R is CH₂CH₃ or CH₃,
   or a pharmaceutically acceptable salt thereof. In certain embodiments, X₃ is D-Lys and R is CH₂CH₃. In certain embodiments, X₂ is Tyr, X₃ is D-Lys, X₄ is Leu, X₅ is Arg and R is CH₂CH₃. In certain embodiments, X₂ is Tyr, X₃ is D-Lys(Ahx), X₄ is Leu, X₅ is Arg and R is CH₂CH₃. In certain embodiments, R is CH₂CH₃. In certain embodiments, R is CH₃. In some embodiments, the invention provides a LHRH peptide conjugate comprising a LHRH peptide derivative comprising the sequence: Gln-His-Trp-Ser-Tyr-D-Lys-Leu-Arg-Pro-NHCH₂CH₃ wherein the LHRH peptide derivative is conjugated to a cytotoxic agent via a self immolative linker.

In some embodiments, the invention provides a LHRH peptide conjugate comprising a LHRH peptide derivative comprising the sequence: pGlu-His-Trp-Ser-Tyr-D-Lys-Leu-Arg-Pro-NHCH₂CH₃ wherein the LHRH peptide derivative is conjugated to a cytotoxic agent via a self immolative linker.

In some embodiments, the invention provides a LHRH peptide conjugate comprising a LHRH peptide derivative comprising the sequence: pGlu-His-Trp-Ser-Tyr-D-Lys(Ahx)-Leu-Arg-Pro-NHCH₂CH₃ wherein the LHRH peptide derivative is conjugated to a cytotoxic agent via a self immolative linker.

Further preferred embodiments of the invention will now be described, by way of example only, with reference to the accompanying drawings.

### EXAMPLES

### Example 1: Synthesis of Different LHRH Peptide Derivatives

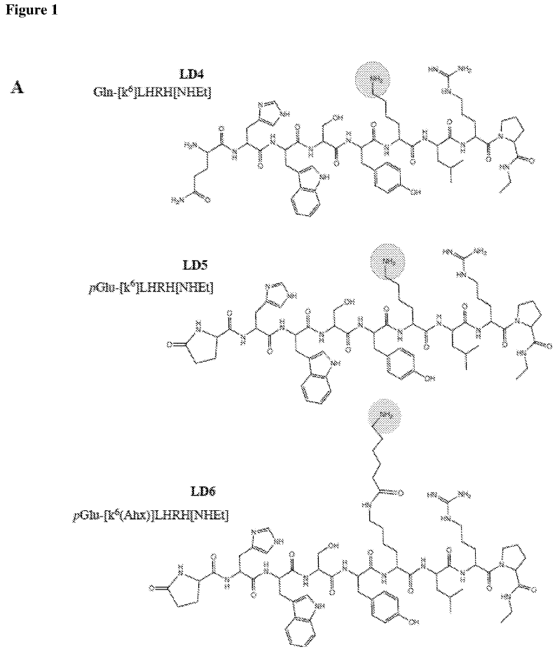

The LHRH peptide derivatives of the invention were designed and synthesized on Rink amide resin following the in situ neutralization protocol (P. Varamini et al., J Med Chem., 2017, 60; P. Varamini et al., Int J Pharm, 2017, 521) for Fmoc solid-phase chemistry (the structure of the LHRH peptide derivatives are shown in FIG. 1A). The purity of each of the peptides (LD4, LD5 and LD6) was greater than 98%. The peptides were purified by reverse phase high performance liquid chromatography (RP-HPLC) on a Shimadzu system using a Vydac C18 column (5 mm, 22 250 mm) running a gradient of two solvents, A: H2O, 0.1% TFA, and B: acetonitrile/H2O 9:1, 0.1% TFA. Either a gradient of 20% to 60% B over 60 min (peptides 1-3, 7 and 9-10) or a gradient of 10% to 60% B over 70 min (peptides 4-6 and 8) was used at a flow rate of 10 mL/min. Collected fractions were analyzed by High resolution MS and ESI-MS and analytical RP-HPLC using a Vydac C4 and C18 column (5 mm, 4.6 250 mm) and a gradient of 0% to 100% B over 30 min at a flow rate of 1 mL/min. Pure fractions were combined and lyophilized

### Example 2: Synthesis of LD5-mc-vc-PABC-MMAE Conjugate

The LD5-me-vc-PABC-MMAE conjugate was synthesized by Fmoc solid-phase chemistry techniques (P. Varamini et al., J Med Chem., 2017, 60; P. Varamini et al., Int J Pharm, 2017, 521).

### Example 3: Metabolic Stability of LHRH Peptide Derivatives

The inventors surprisingly found that the LHRH peptide derivatives have improved metabolic stability. The metabolic stability of the native LHRH peptide, LHRH peptide derivatives LD4, LD5 and LD6, and known LHRH agonists triptorelin ([w6]LHRH) and [k6]LHRH were examined in human plasma (FIG. 1B). The LHRH peptide derivatives LD4, LD5 and LD6 exhibit substantially improved half-life (T_{1/2} of about 257 min, 365 min and 309 min for LD-4, LD-5 and LD-6, respectively) compared to the native LHRH peptide (T_{1/2} of about 10 min) and LHRH agonists that are known in the art, i.e. triptorelin ([w⁶] LHRH) and [k⁶] LHRH (T_{1/2} of about 19 min and 39 min, respectively).

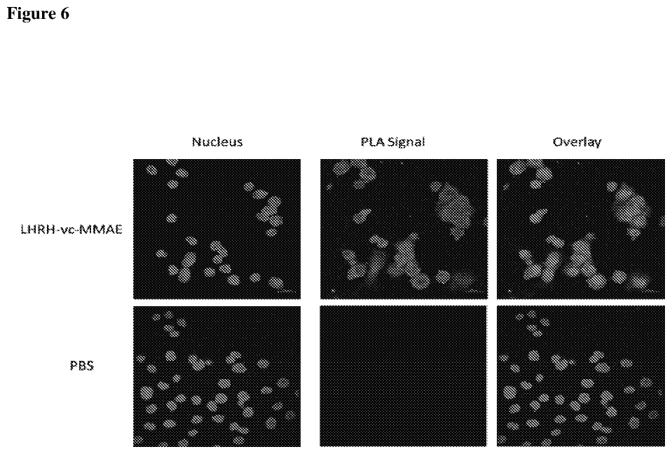

LD4, LD5 and LD6 show uniquely high stability but among all, LD5 had the highest half-life of 365 min and was selected for conjugation to MMAE via the self-immolative linker, mc-vc-PABC. The binding affinity of the LD5-me-vc-PABC-MMAE conjugate to LHRH receptors was investigated by DuoLink assay. The assay, as shown in FIG. 6, confirmed a high binding affinity of the peptide ligand to LHRH receptors.

### Example 4: Anti-Proliferative Activity

The inventors further discovered that LD4, LD5 and LD6 have significantly higher anti-proliferative activities in 3 different breast cancer cell lines compared to both native LHRH and the agonist [w6]LHRH, which has been used in the clinic for hormone-dependent gynaecological cancers (see Table 1).

### Example 5: LHRH Receptor (LHRH-R) is Expressed in Human Breast Cancer Cell Lines

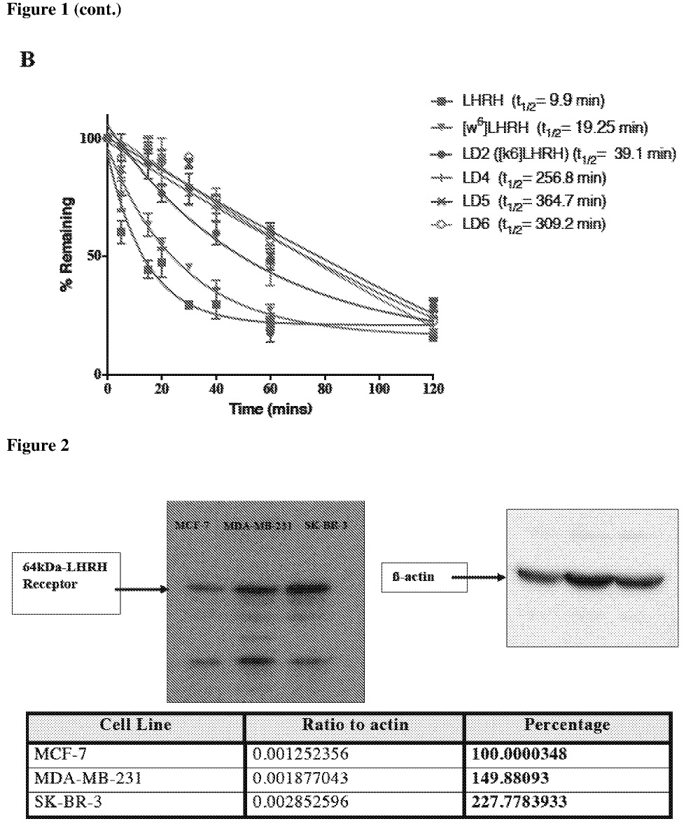

Cultures from breast cancer cells MDA-MB-231, SK-BR-3 and MCF-7 were harvested and lysed in lysis buffer (150 mM NaCl, 1% Triton X-100, 0.1% SDS, 50 mM TrisHCl, pH8.0, and protease inhibitor mixture), and sonicated 15 times for 1 second on ice, followed by centrifugation at 16,100×g at 4° C. for 30 minutes. 50 micrograms of total protein extracts were subjected to 8% SDS/PAGE gel. Following overnight transfer at 4° C., polyvinylidene difluoride (PDVF) membranes were blocked in 5% bovine serum albumin (BSA) for 1 hour, and incubated with anti-LHRH receptor primary antibodies (SolarBio Life Sciences) overnight at 4° C. After washing in Tris buffered saline with Tween (TBS-T), membranes were incubated with horseradish peroxidase-conjugated (HRP-conjugated) secondary antibody for 1 hour. After washing, proteins were detected using ECL-Plus chemiluminescence detection system (GE Healthcare). Density was measured using the Image J program. β-actin was used as control in Western Blot to determine LHRH-R expression in MDA-MB-231, SK-BR-3 and MCF-7 cancer cell lines. Western blot analysis indicated expression of LHRH receptors in 3 breast cancer cell lines with different characteristics (MDA-MB-231: ER−, HER-2−, PR−; MCF-7, ER+, HER-2+, PR+; and SK-BR-3, ER−, HER-2+, PR−). These cell lines were used for the anti-proliferative studies of the LHRH peptide derivatives. This study was performed using the antibody specifically raised against the LHRH-R. A major protein band of approximately 64 kDa molecular mass known for the human pituitary LHRH receptor was identified in MCF-7, MDA-MB-231 and SK-BR-3 (FIG. 2 lanes 1, 2, and 3 respectively). The level of LHRH-R expression relative to 3-actin in MCF-7, MDA-MB-231 and SK-BR-3 showed that these breast cancer cells can be actively targeted through the LHRH receptor.

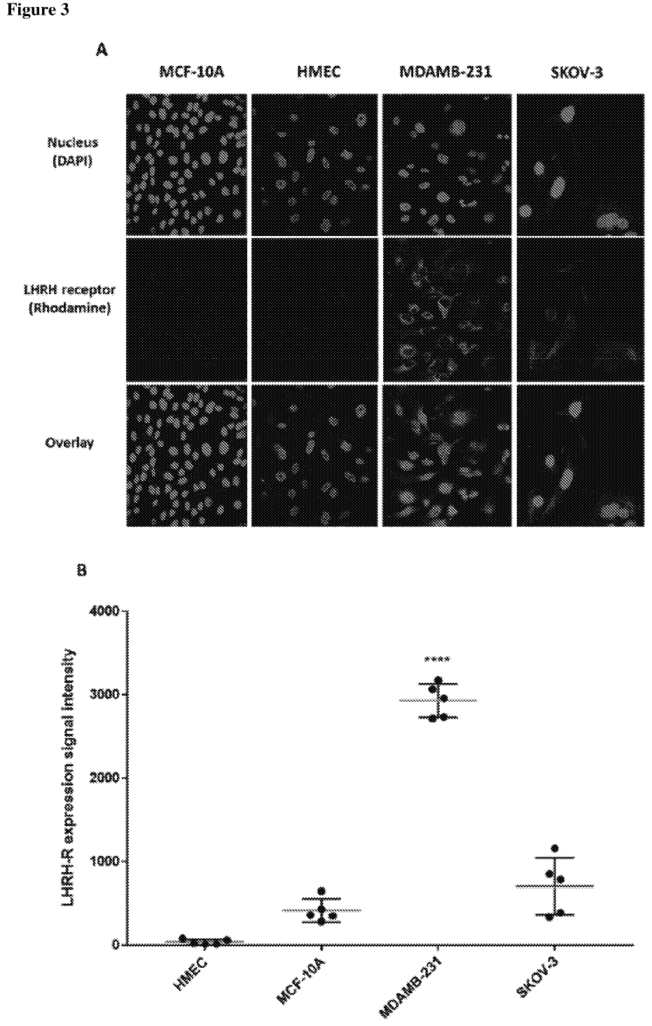

Immunocytochemistry was used to confirm the expression of LHRH-Rs in MDA-MB-231 (TNBC cell model), SKOV-3 (low LHRH-R expressing cancer cell model) as well as HMEC and MCF-10A (i.e. normal breast cells) (FIG. 3A). The quantification analysis (FIG. 3B) showed LHRH-R signal intensity is significantly higher in MDA-MB-231 (TNBC cell model) compared to SKOV-3 (low-expressing LHRH-R cancer cell model) and normal breast cells (p<0.05).

TABLE 1
Direct antiproliferative activity of LHRH peptide derivatives

| Cell | | | | LHRH | [w⁶]GnRH |
|---|---|---|---|---|---|
| Line | LD4 | LD5 | LD6 | (9) | (10) |
| MDA-MB- | 36.2 ± 0.8* | 38.1 ± 1.4* | 33.2 ± 2.5* | >100 | 68.1 ± 8.1 |
| 231 (μM) | | | | | |
| MCF-7 | 51.3 ± 3.1* | 47.6 ± 5.4* | 49.2 ± 2.1* | >100 | 79.4 ± 5.3 |
| (μM) | | | | | |
| SK-BR-3 | 31.2 ± 4.8* | 33.5 ± 2.5* | 29.7 ± 1.1* | >100 | 81.2 ± 7.2 |
| IC₅₀ (μM) | | | | | |
| SKOV-3 | >100 | >100 | >100 | >100 | >100 |
| IC₅₀ (μM) | | | | | |

The IC₅₀ values (μM) were estimated from concentration-response curves using non-linear regression for inhibition of cell growth. Data are expressed as mean±SD from at least three independent experiments, each in triplicate. Statistical analysis was performed using a two-way ANOVA (* p<0.05, the IC₅₀ for each compared with that of their corresponding parent peptide for the same cell line).

### Example 6: Cytotoxicity of LD5-mv-vc-PABC-MMAE

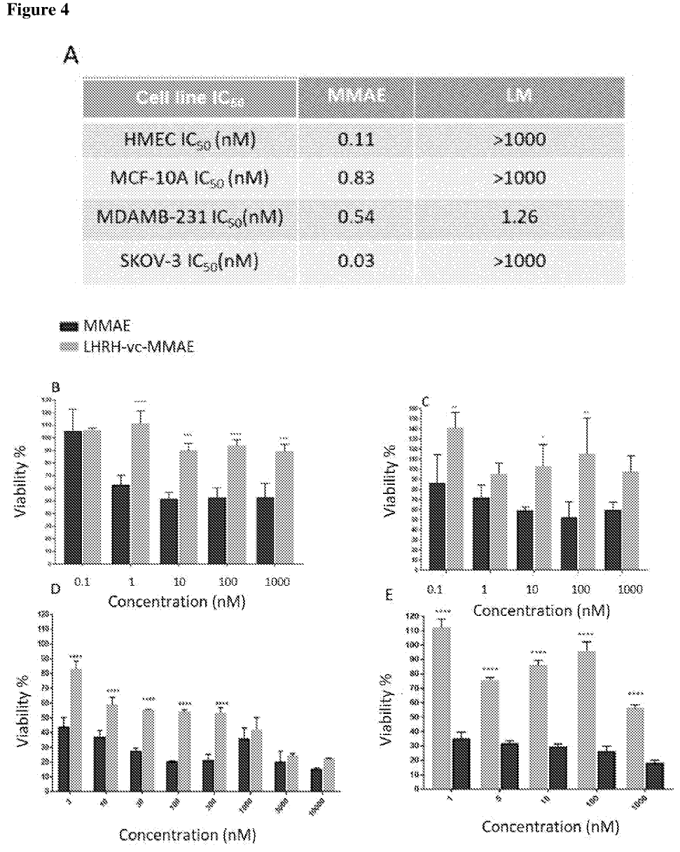

The cytotoxicity of the LHRH peptide derivative pGlu-His-Trp-Ser-Tyr-D-Lys-Leu-Arg-Pro-NHCH₂CH₃ (LD5) conjugated with MMAE by the self-immolative linker, mv-vc-PABC (i.e. LD5-mv-vc-PABC-MMAE) was determined with the MMT assay. This assay was used to examine whether LD5-mv-vc-PABC-MMAE affects the proliferation of TNBC cells that expresses LHRH-R. The TNBC cell model, MDA-MB-231, was used to screen for the relative cytotoxicity of LD5-me-vc-PABC-MMAE compared to MMAE and in normal breast cells, HMEC and MCF-10A as well as SKOV-3 (LHRH-R negative controls). Cells were incubated with each compound for 72 h and relative cellular viability was determined using the colorimetric MTT assay. The growth inhibitory effect of LD5-me-vc-PABC-MMAE and MMAE was reported as IC₅₀ (nM) values for normal breast cells, MDA-MB-231 and SKOV-3 (FIG. 4A).

Normal breast cells (HMEC and MCF-10A) were sensitive to MMAE with IC₅₀ value of 0.11 nM and 0.83 nM, respectively. However, LD5-me-vc-PABC-MMAE did not show significant cytotoxic effect on normal breast cells (IC₅₀ value of >1000 nM). In contrast, the TNBC cell line (MDA-MB-231) showed significant sensitivity to LD5-me-vc-PABC-MMAE with IC₅₀ value of 1.26 nM. Although the SKOV-3 cell line had higher sensitivity to MMAE with IC₅₀ value of 0.03 nM, this cell line was resistant to LD5-me-vc-PABC-MMAE with IC₅₀ value of >1000 nM.

The cytotoxicity of LD5-mc-vc-PABC-MMAE was assessed in comparison to MMAE for all cells. The potency of LD5-me-vc-PABC-MMAE was significantly lower than MMAE in certain concentrations in normal breast cells (HMEC and MCF-10A), TNBC cells (MDA-MB-231) and LHRH-R negative controls (SKOV-3) (FIGS. 4B, 4C, 4D and 4E). This decrease in cytotoxicity of MMAE when conjugated with LD5 demonstrates the selectivity of the LHRH uptake pathway via the LHRH receptor in comparison to simple diffusion of free MMAE in the cell lines.

### Example 7: Role of LHRH Receptor in Cytotoxicity of LD5-mc-vc-PABC-MMAE

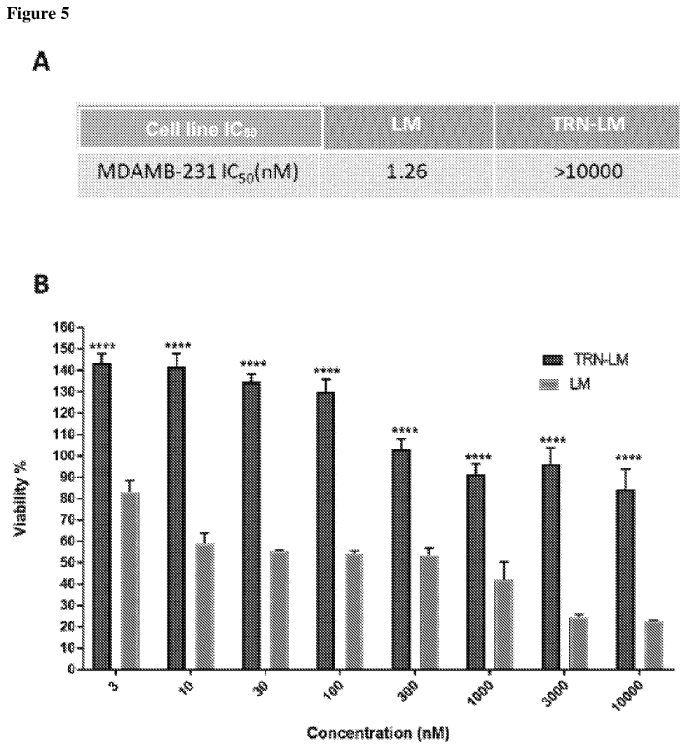

Receptor binding competitive assay was performed to examine the association of cytotoxicity with binding to LHRH-Rs. TNBC cells (MDA-MB-231) were pre-treated with 100 M of triptorelin (TRN) for 2 h to block the LHRH-R. The significant cytotoxic effects of LD5-mc-vc-PABC-MMAE (LM) was reversed after pre-treatment with TRN (FIGS. 5A and 5B). The MTT assay showed that blocking LHRH-R through pre-treatment with TRN significantly increases the cell viability of LD5-me-vc-PABC-MMAE treated cells. This demonstrates the significant role that LHRH-Rs play in LD5-me-vc-PABC-MMAE's anti-cancer activity (FIG. 5).

### Example 8: Interaction of LHRH-R and LHRH in LD5-mc-vc-PABC-MMAE Treated Environment

Proximity ligation assay (PLA) was used to determine the interaction between LHRH-R and LHRH in LD5-mc-vc-PABC-MMAE treated cells. Duolink® Proximity Ligation Assay (PLA) allows in situ detection of endogenous proteins, protein modifications, and protein interactions with high specificity and sensitivity. Protein targets can be readily detected and localized with single molecule resolution in unmodified cells and tissues. Typically, two primary antibodies raised in different species are used to detect two unique protein targets. PLA reagents were added to the fixed MDA-MB-231 cells after incubating these cells with the primary antibody specific for LHRH-R and LHRH. In cells treated with LD5-me-vc-PABC-MMAE, the signal localization reveals the protein interaction to be at intracellular sites as well as at the plasma membrane (FIG. 6). This qualitative result confirmed interactions between LHRH-R and its ligand during the LD5-me-vc-PABC-MMAE uptake by MDA-MB-231 cells.

### Example 9: In-Vitro Uptake of LD5-mc-vc-PABC-MMAE in TNBC Cells

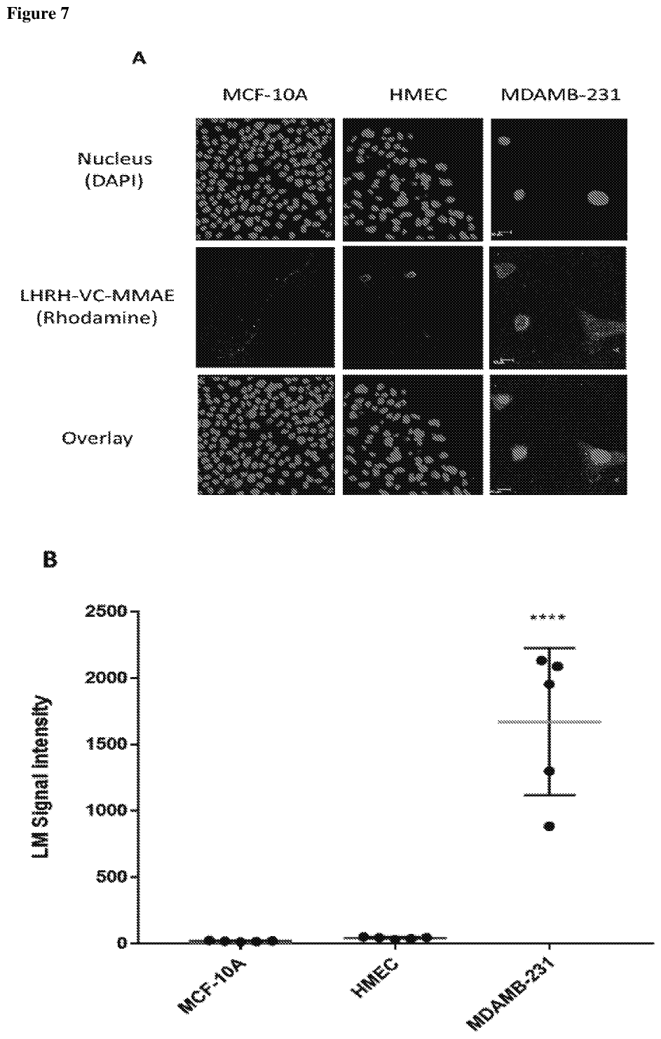

The uptake of LD5-me-vc-PABC-MMAE by TNBC cells was examined using the TNBC cell line (MDA-MB-231) which overexpresses LHRH-R. The uptake was compared with normal breast cells (LHRH-R negative control). LD5-me-vc-PABC-MMAE was incubated with MDA-MD-231 normal breast cells for 18 h, and intracellular uptake of the conjugate was monitored using confocal LSM (FIG. 7A).

As shown in FIG. 7A, there was a significantly higher uptake of LD5-mc-vc-PABC-MMAE in LHRH-R positive cells (MDA-MB-231) compared to normal breast cells (MCF-10A and HMEC). This was supported by quantitative comparisons of the intracellular uptake of LD5-mc-vc-PABC-MMAE in LHRH-R positive and negative cells (FIG. 7B). These data support the active targeted delivery of the compound through LHRH-R in TNBC cells (p<0.05).

### Example 10: Effects of LD5-mc-vc-PABC-MMAE on α-Tubulin Polymerisation in Normal and Cancer Cells

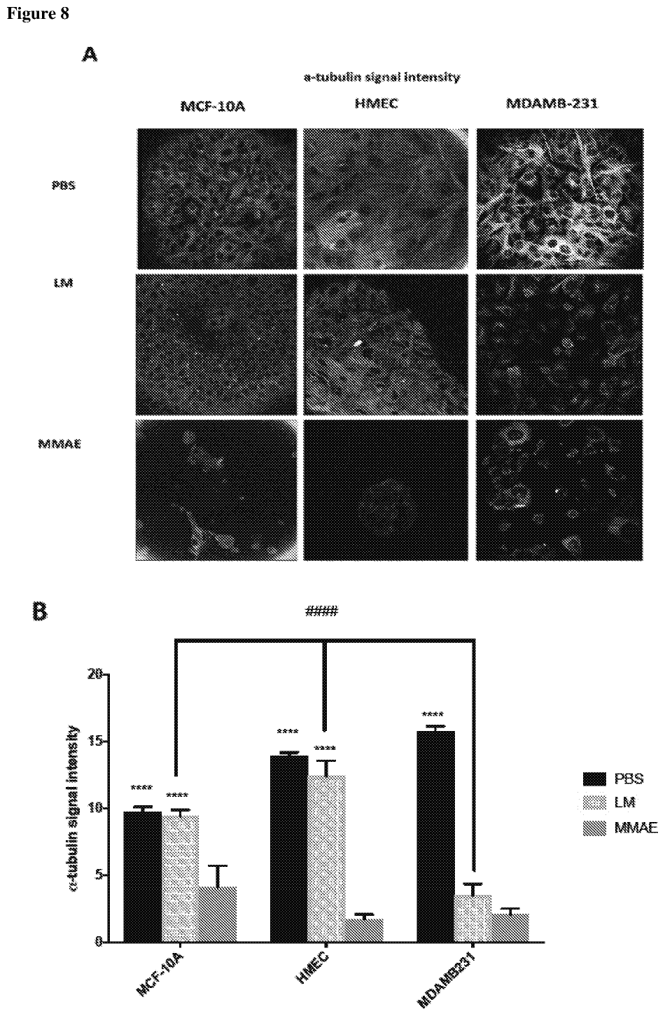

The intracellular effects of MMAE on α-tubulin polymerisation were examined using α-tubulin immunostaining in TNBC cells (MDA-MB-231) and normal breast cells (MCF-10A and HMEC) treated with MMAE and MMAE conjugated with LD5 (LD5-mc-vc-PABC-MMAE). TNBC cells and normal breast cells were fixed after incubating with 1 M of MMAE and LD5-mc-vc-PABC-MMAE for 18 h along with PBS as a control. The α-tubulin was stained by immunostaining and observed by confocal LSM. As shown in FIG. 8A, TNBC cells and normal breast cells treated with MMAE show dramatic decrease in α-tubulin formation compared to PBS control indicating its non-selective activity against normal breast cells and cancer cells (i.e. TNBC cell model). The α-tubulin signal in normal breast cells treated with PBS and LD5-mc-vc-PABC-MMAE (LM) were significantly higher than their MMAE treatment counterpart group (FIG. 8B) indicating the lower impact of the targeted drug on the α-tubulin polymerisation as a factor of cell survival (FIG. 4).

### Example 11: Effects of Silencing LHRH-R Gene on the Uptake of LD5-mc-vc-PABC-MMAE by TNBC Cells

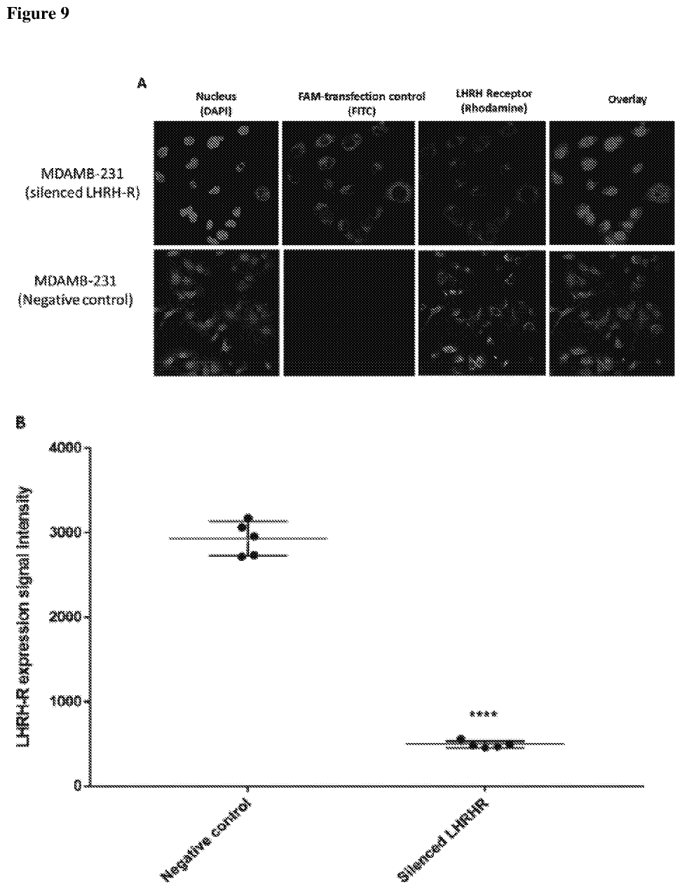

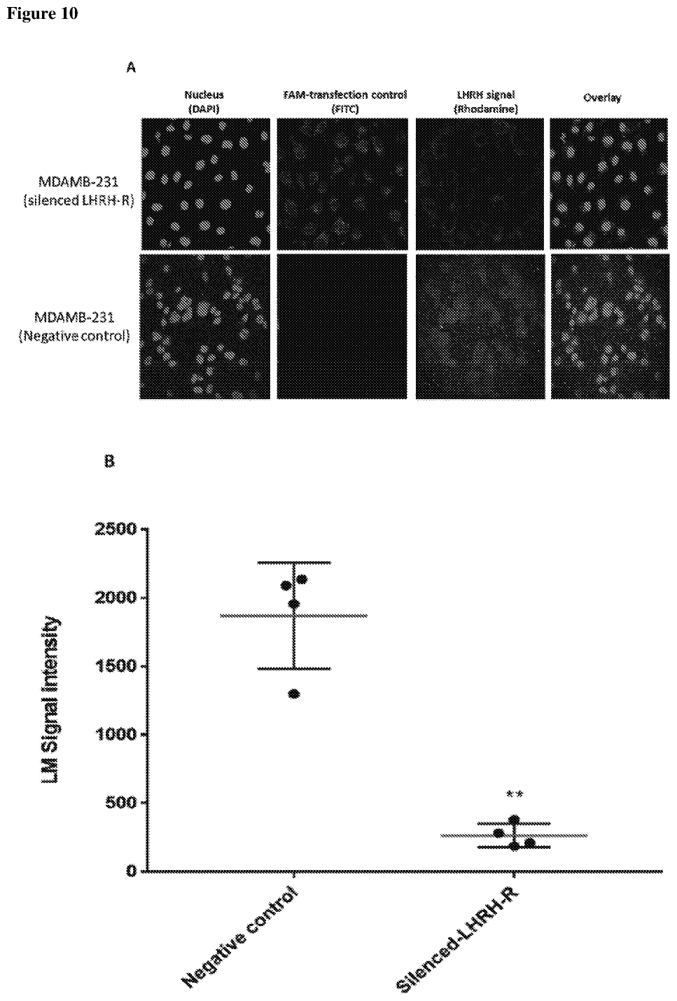

To further examine the role of LHRH-R in the uptake of LD5-me-vc-PABC-MMAE, LHRH-R expression was blocked by silencing their gene in MDA-MB-231 (TNBC cell model). This study was performed by co-transfection of siRNAs (RNAi-Mate transfection reagent and siRNA, GNRHR-homo-2242, GNRHR-homo-2701, and scrambled RNA were from GenePharma) and a fluorescently labelled negative control siRNA (FAM transfection efficiency control was from GenePharma). As shown in FIG. 9, silencing yield over 83% reduction in LHRH-R expression in transfected MDA-MB-231 cells compared to negative control (FIG. 9, p<0.05). The uptake of LD5-me-vc-PABC-MMAE was significantly reduced after silencing the LHRH-R gene expression indicating that the construct is actively targeted to be up-taken by the cancer cells via the overexpressed LHRH-Rs (FIG. 10, p<0.05).

### Example 12: In Vitro Metabolic Stability of LD5-(mc-vc-PABC)-MMAE

The metabolic stability of LD5-(mc-vc-PABC)-MMAE was investigated in cell culture media (c-media), human and mouse plasma. An LC/MS method was developed to detect both free MMAE and the presence of any degraded species from the whole construct, LD5-(mc-vc-PABC)-MMAE. To evaluate the stability of the valine-citrulline linkage, LD5-(mc-vc-PABC)-MMAE at 1 μM was incubated in c-media, human and mouse plasma at 37° C. for a period of 10 days. Aliquots were taken at pre-determined time intervals (t=0, 1, 2, 4, 7, 10 days) and analysed by LC/MS for the release of free MMAE.

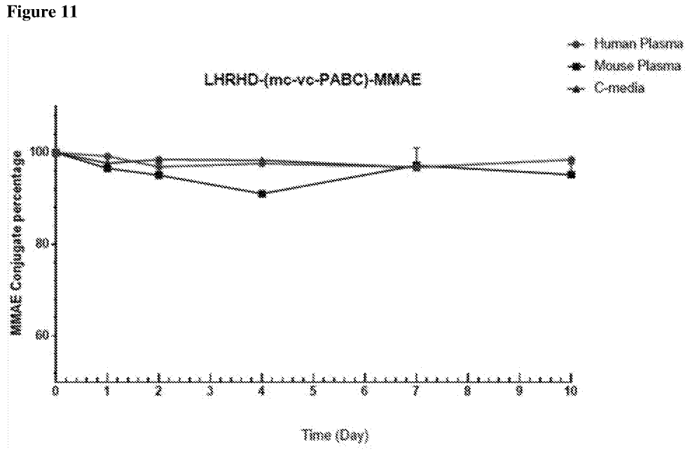

It was shown that LD5-(mc-vc-PABC)-MMAE remained stable during the course of the study, which was 10 days (FIG. 11). This study revealed that less than 3% of the total drug was released as MMAE in human plasma and c-media after 10 days. In mouse plasma, less than 5% of the total drug was released after 10 days. Furthermore, qualitative full-scan LC/MS analysis, including UV detection, of the same samples did not reveal the presence of other molecular species identifiable as drug or drug-linker degradation products.

The use of a highly stable peptide derivative and an intracellular linker in the design on LHRHD-(mc-vc-PABC)-MMAE (i.e. LD-5-(mc-vc-PABC)-MMAE) resulted in a stable conjugate in human and mouse plasma as well as cell culture media (Example 12). The drug was stably attached to the peptide, showing only around 3% release of MMAE following 10-day incubation in human plasma, but in vitro data showed that it had been cleaved by lysosomal proteases once internalised via LHRH receptors. These stability data were comparable with those of ADCs in the clinic containing MMAE such as Brentuximab vedotin (cAC10-vcMMAE) or Trastuzumab Ermtansine (T-DMI) (J A. Francisco et al., Blood, 2003, 102; B. Bender et al., The AAPS Journal, 2014, 16). While these conjugates with these stability data have been successful in the clinical trials and they are in the market now, there is no report on the in vitro stability of Zoptarelin Doxorubicin (AEZS 108, an LURH receptor-targeted PDC that reached clinical trials). The reason this conjugate failed in phase III clinical trials was lack of stability and release of doxorubicin in the plasma before reaching the tumour site. This led the PDC to have a similar safety profile as that of free doxorubicin. There was no superiority in both toxicity and efficacy over free doxorubicin.

### Example 13: In Vivo Minimum Tolerated Dose and Toxicity Studies

A. Phase I: Single-Dose MTD

LD5-(mc-vc-PABC)-MMAE was administered IV to groups of 3 female NOD/SCID mice (23±3 g). Animals received an initial dose of 3 mg/kg. If the animals survived for 72 hours, the dose for the next cohort was increased. If one or more animals died, the dose for the next cohort was decreased. The testing stopped when all animals survived at the upper bound, or when two or three dose levels had been tested or when the upper or lower bound had been reached. At each dose level, animals were observed for the presence of acute toxic symptoms (mortality, convulsions, tremors, muscle relaxation, sedation, etc.) and autonomic effects (diarrhea, salivation, lacrimation, vasodilation, piloerection, etc.) during the first 15 minutes then again at 1 and 2 hours. Body weights were recorded pre-dose and at 72 hours after treatment. The animals were observed and mortality noted daily after compound administration for 3 days. Gross necropsy was performed on all animals without tissue collection. No significant adverse effects were noted in both 3 and 10 mg/kg through IV injection at all monitored time points (15 minutes, 1 and 2 hours). No mortality and body weight changes were noted, signifying the dose level was tolerated (Tables 2 and 3). The dose at 10 mg/kg was then determined for the following repeat-dose MTD study (Phase II).

TABLE 2
Maximum Tolerated Dose, Autonomic Signs in Mice (Phase I: Mortality)

| | | Dose | Mortality (death/test) | | | | | |
|---|---|---|---|---|---|---|---|---|
| Compound | Route | (mg/kg) | 15 min | 1 hr | 2 hr | 24 hr | 48 hr | 72 hr |
| Vehicle | IV | 5 mL/kg | 0/3 | 0/3 | 0/3 | 0/3 | 0/3 | 0/3 |
| (PBS) | | | | | | | | |
| LHRHD-(mc- | IV | 3 | 0/3 | 0/3 | 0/3 | 0/3 | 0/3 | 0/3 |
| vc-PABC)- | | 10 | 0/3 | 0/3 | 0/3 | 0/3 | 0/3 | 0/3 |
| MMAE | | | | | | | | |

TABLE 3
Maximum Tolerated Dose, Autonomic Signs in Mice
(Phase I: Body weight)

| | | Dose | | Body Weight (g) | |
|---|---|---|---|---|---|
| Compound | Route | (mg/kg) | No. | 0 | 72 hr |
| Vehicle | IV | 5 | 1 | 22 | 22 |
| (PBS) | | mL/kg | 2 | 22 | 22 |
| | | | 3 | 21 | 21 |
| LHRHD-(mc- | IV | 3 | 1 | 21 | 21 |
| vc-PABC)- | | | 2 | 21 | 21 |
| MMAE | | | 3 | 21 | 21 |
| | | 10 | 1 | 20 | 21 |
| | | | 2 | 20 | 20 |
| | | | 3 | 20 | 20 |

B. Phase II: Repeat-Dose MTD

LD-5-(mc-vc-PABC)-MMAE (10 mg/kg; determined by the results by Phase I) was administered IV once weekly on days 1 and 8 to groups of 3 female NOD/SCID mice (23±3 g). Animals were observed for the presence of acute toxic symptoms (mortality, convulsions, tremors, muscle relaxation, sedation, etc.) and autonomic effects (diarrhea, salivation, lacrimation, vasodilation, piloerection, etc.) during the first 15 minutes then again at 1 and 2 hours after each treatment on days 1 and 8. Body weights were recorded pre-dose and on days 1, 4, 8, 12 and 15. The animals were observed and mortality noted daily after first compound administration for 15 days. Gross necropsy was performed on all animals without tissue collection. No marked adverse effects were observed at 10 mg/kg IV after the first and the second administrations of LD-5(mc-vc-PABC)-MMAE on Day 1 and 8 (15 minutes, 1 and 2 hours); in addition, all of the tested animals were alive at the termination of the study period, suggesting the dose level were tolerated after the repeat administrations on days 1 and 8 (Tables 3 and 4). No abnormality was found after the gross necropsy in both phases.

TABLE 4
Maximum Tolerated Dose, Autonomic Signs in Mice - Phase II Mortality

| | | Dose | Mortality (death/test) | | | | | | | |
|---|---|---|---|---|---|---|---|---|---|---|
| Compound | Route | (mg/kg) | Day 1 | Day 2 | Day 3 | Day 4 | Day 5 | Day 6 | Day 7 | Day 8 |
| Vehicle | IV | 5 mL/kg | 0/3 | 0/3 | 0/3 | 0/3 | 0/3 | 0/3 | 0/3 | 0/3 |
| (PBS) | | | | | | | | | | |
| LHRHD-(mc- | IV | 10 | 0/3 | 0/3 | 0/3 | 0/3 | 0/3 | 0/3 | 0/3 | 0/3 |
| vc-PABC)- | | | | | | | | | | |
| MMAE | | | | | | | | | | |

| | | Dose | Mortality (death/test) | | | | | | |
|---|---|---|---|---|---|---|---|---|---|
| Compound | Route | (mg/kg) | Day 9 | Day 10 | Day 11 | Day 12 | Day 13 | Day 14 | Day 15 |
| Vehicle | IV | 5 mL/kg | 0/3 | 0/3 | 0/3 | 0/3 | 0/3 | 0/3 | 0/3 |
| (PBS) | | | | | | | | | |
| LHRHD-(mc- | IV | 10 | 0/3 | 0/3 | 0/3 | 0/3 | 0/3 | 0/3 | 0/3 |
| vc-PABC)- | | | | | | | | | |
| MMAE | | | | | | | | | |

TABLE 5
Maximum Tolerated Dose, Autonomic Signs in Mice - Phase II Body Weight

| | | Dose | | Body Weight (g) | | | | |
|---|---|---|---|---|---|---|---|---|
| Compound | Route | (mg/kg) | No. | Day 1 | Day 4 | Day 8 | Day 12 | Day 15 |
| Vehicle | IV | 5 mL/kg | 1 | 21 | 23 | 24 | 25 | 26 |
| (PBS) | | | 2 | 23 | 23 | 23 | 23 | 25 |
| | | | 3 | 22 | 23 | 24 | 25 | 26 |
| LHRHD-(mc- | IV | 10 | 1 | 22 | 22 | 22 | 23 | 24 |
| vc-PABC)- | | | 2 | 22 | 23 | 23 | 24 | 25 |
| MMAE | | | 3 | 21 | 23 | 23 | 24 | 25 |

The MTD and toxicity studies of LHRHD-(me-v-PABC)-MMAE (i.e. LD-5-(mc-vc-PABC)-MMAE) was performed in female NOD/SCID mice, and no toxicity was observed in any of the mice being dosed at 10 mg/kg. Therefore, the MTD was not reached at this dose. With the corresponding ADCs bearing MMAE the MTD has been considerably lower. For example, MTD for brentuximab vedotin in mice was achieved at 30-40 mg/kg which is equivalent to approximately 70 mg/kg of LHRHD-(mc-vc-PABC)-MMAE. Considering the comparable selectivity and stability of LHRHD-(mc-vc-PABC)-MMAE with the corresponding ADCs, and a markedly lower MTD, a superior safety profile is predicted for this PDC. Other advantages for this PDC over current ADCs are a significantly lower cost of production and simpler manufacturing processes.

1. A LHRH peptide-drug conjugate (LHRH-PDC) comprising the sequence:
X₁-His-Trp-Ser-X₂-X₃(L-D)-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys;
R is CH₂CH₃ or CH₃;
wherein
L is a linker; and
D is a cytotoxic agent,
or a pharmaceutically acceptable salt thereof.

2.-4. (canceled)

5. The LHRH-PDC of claim 1, wherein X₁ is Gln, X₂ is Tyr, X₃ is D-Lys, X₄ is Leu, X₅ is Arg and R is CH₂CH₃.

6. The LHRH-PDC of claim 1, wherein X₁ is pGlu, X₂ is Tyr, X₃ is D-Lys, X₄ is Leu, X₅ is Arg and R is CH₂CH₃.

7. The LHRH-PDC of claim 1, wherein X₁ is pGlu, X₂ is Tyr, X₃ is D-Lys(Ahx), X₄ is Leu, X₅ is Arg and R is CH₂CH₃.

8.-20. (canceled)

21. The LHRH-PDC of claim 1, wherein the linker is a self-immolative linker.

22. The LHRH-PDC of claim 1, wherein the linker is maleimidocaproyl valine-citrulline-p-aminobenzyl carbamoyl (mc-vc-PABC).

23. The LHRH-PDC of claim 1, wherein the cytotoxic agent is an anti-mitotic agent, an alkylating agent, an anti-metabolite, a topoisomerase inhibitor or a protein kinase inhibitor.

24. The LHRH-PDC of claim 1, wherein the cytotoxic agent is a vinca alkaloid, a cryptophycin, bortezomib, thiobortezomib, a tubulysin, aminopterin, rapamycin, paclitaxel, docetaxel, daunorubicin, everolimus, a-amanatin, vemcarin, didemnin B, geldanomycin, purvalanol A, ispinesib, budesonide, dasatinib, an epothilone, a maytansine, doxorubicin, camptothecin, methotrexate (MTX) or monomethyl auristatin E (MMAE).

25. The LHRH-PDC of claim 1, wherein the cytotoxic agent is MMAE.

26. An anti-proliferative LHRH peptide derivative comprising the sequence:
X₁-His-Trp-Ser-X₂-X₃-X₄-X₅-Pro-NHR
wherein
X₁ is pGlu or Gln;
X₂ is Tyr, Phe or His;
X₃ is D-Lys or D-Lys(Ahx);
X₄ is Leu, Val, Trp or Met;
X₅ is Arg, Gln, Trp, Ser, Leu, Asn, Phe, Tyr or Lys; and
R is CH₂CH₃ or CH₃,
or a pharmaceutically acceptable salt thereof: wherein
X₁ is Gln, X₂ is Tyr, X₃ is D-Lys, X₄ is Leu, X₅ is Arg and R is CH₂CH₃;
X₁ is pGlu, X₂ is Tyr, X₃ is D-Lys, X₄ is Leu, X₅ is Arg and R is CH₂CH₃: or X₁ is pGlu, X₂ is Tyr, X₃ is D-Lys(Ahx), X₄ is Leu, X₅ is Arg and R is CH₂CH₃.

27.-54. (canceled)

55. The anti-proliferative LHRH peptide derivative of claim 26, wherein the LHRH peptide derivative is conjugated to a cytotoxic agent via a self immolative linker.

56.-60. (canceled)

61. A pharmaceutical composition comprising a LHRH-PDC of claim 1 or a pharmaceutically acceptable salt thereof, and optionally at least one pharmaceutically acceptable excipient.

62. A method of treating a cancer in a subject, comprising administering to the subject the pharmaceutical composition of claim 61, wherein the subject has a LHRH receptor expressing cancer.

63. The method of claim 62, wherein the subject has a LHRH receptor expressing cancer selected from the group consisting of breast cancer, a hormone sensitive and/or refractory breast cancer, prostate cancer, colon cancer, ovarian cancer, pancreatic cancer and endometrial cancer.

64. The method of claim 62, wherein the subject has a LHRH receptor expressing cancer, wherein the LHRH receptor expressing cancer is Triple Negative Breast Cancer (TNBC).

65.-73. (canceled)

74. The method of claim 62, wherein:
X₁ is Gln, X₂ is Tyr, X₃ is D-Lys, X₄ is Leu, X₅ is Arg and R is CH₂CH₃;
X₁ is pGlu, X₂ is Tyr, X₃ is D-Lys, X₄ is Leu, X₅ is Arg and R is CH₂CH₃; or
X₁ is pGlu, X₂ is Tyr, X₃ is D-Lys(Ahx), X₄ is Leu, X₅ is Arg and R is CH₂CH₃.

75. The method of claim 62, wherein the cytotoxic agent is MMAE LHRH-PDC and the linker is mc-vc-PABC.

76. The LHRH-PDC of claim 1, comprising the sequence Gln-His-Trp-Ser-Tyr-D-Lys-Leu-Arg-Pro-NHCH₂CH₃ or a pharmaceutically acceptable salt thereof, wherein the cytotoxic agent is MMAE and the linker is maleimidocaproyl valine-citrulline-p-aminobenzyl carbamoyl (mc-vc-PABC), optionally with at least one pharmaceutically acceptable excipient.

77. The LHRH-PDC of claim 1, comprising the sequence pGlu-His-Trp-Ser-Tyr-D Lys-Leu-Arg-Pro-NHCH2CH3 or a pharmaceutically acceptable salt thereof, wherein the cytotoxic agent is MMAE and the linker is maleimidocaproyl valine-citrulline-p-aminobenzyl carbamoyl (mc-vc-PABC), optionally with at least one pharmaceutically acceptable excipient.

78. The LHRH-PDC of claim 1, comprising the sequence pGlu-His-Trp-Ser-Tyr-D Lys(Ahx)-Leu-Arg-Pro-NHCH2CH3 or a pharmaceutically acceptable salt thereof, wherein the cytotoxic agent is MMAE and the linker is maleimidocaproyl valine-citrulline-p-aminobenzyl carbamoyl (mc-vc-PABC), optionally with at least one pharmaceutically acceptable excipient.
